(19) United States
(12) Patent Application Publication
(10) Pub. No.: US 2005/0215641 A1
(43) Pub. Date: Sep. 29, 2005

(54) Compositions comprising reverse isomers of conjugated linoleic acid

(76) Inventors: Asgeir Saebo, Eidsnes (NO); Per Christian Saebo, Volda (NO); Mikko Griinari, Espoo (FI); Dale E. Bauman, Ithaca, NY (US); Kevin Shingfield, Ypaja (FI)

Correspondence Address: J. Mitchell Jones;MEDLEN & CARROLL, LLP, Suite 350, 101 Howard Street, San Francisco, CA, 94105, US

(21) Appl. No.: 11/077,044

(22) Filed: Mar. 10, 2005

Related U.S. Application Data
(60) Provisional application No. 60/551,983, filed on Mar. 10, 2004.

(51) Int. Cl. A61K 31/202; C07C 51/347

(52) U.S. Cl. 514/560; 554/219

(57) ABSTRACT

The present invention relates to the field of human and animal nutrition, and in particular to certain novel compositions of conjugated linoleic acids (CLA). In particular, the present invention relates to CLA compositions comprising the c10,t12, c10,c12, t9,c11 and c9,c11 isomers of conjugated linoleic acid.

This application claims priority to Provisional Appl. 60/551,983 filed Mar. 10, 2004.

### FIELD OF THE INVENTION

The present invention relates to the field of human and animal nutrition, and in particular to certain novel compositions of conjugated linoleic acids (CLA). In particular, the present invention relates to CLA compositions comprising the c10,t12, c10,c12, t9,c11 and c9,c11 isomers of conjugated linoleic acid.

### BACKGROUND OF THE INVENTION

In 1978, researchers at the University of Wisconsin discovered that cooked beef contained a substance that appeared to inhibit mutagenesis. Later the substance was found to be a mixture of positional isomers of linoleic acid (C18:2) having conjugated double bonds. The c9,t11 and t10,c12 isomers are present in greatest abundance in the materials that were used in the early studies, but it is uncertain which isomers are responsible for the biological activity observed. It has been noted from labeled uptake studies that the 9,11 isomer appears to be somewhat preferentially taken up and incorporated into the phospholipid fraction of animal tissues, and to a lesser extent the 10,12 isomer. (Ha, et al., Cancer Res., 50: 1097 [1990]).

The biological activity associated with conjugated linoleic acids (termed CLA) is diverse and complex. At present, very little is known about the mechanisms of action, although several preclinical and clinical studies in progress are likely to shed new light on the physiological and biochemical modes of action. The anticarcinogenic properties of CLA have been well documented. Administration of CLA inhibits rat mammary tumorigenesis, as demonstrated by Ip, et al. Cancer Res., 51:6118 [1991]. Ha, et al., Cancer Res., 50: 1097 [1990] reported similar results in a mouse forestomach neoplasia model. CLA has also been identified as a strong cytotoxic agent against target human melanoma, colorectal and breast cancer cells in vitro. A recent major review article confirms the conclusions drawn from individual studies (Ip, Am. J. Clin. Nutr., 66 (6 Supp): 1523s [1997]).

Another important source of interest in CLA, and one which underscores its early commercial potential, is that it is naturally occurring, especially the c9,t11 isomer, in foods and feeds consumed by humans and animals alike. In particular, CLA is abundant in products from ruminants. For example, several studies have been conducted in which CLA has been surveyed in various dairy products. Aneja, et al., J. Dairy Sci., 43: 231 [1990] observed that processing of milk into yogurt resulted in increased concentration of CLA. Shantha, et al., Food Chem., 47: 257 [1993] showed that a combined increase in processing temperature and addition of whey increased CLA concentration during preparation of processed cheese.

As a result, the majority of studies on CLA thus far have focused on the effects of two isomers: the c9,t11 isomer and the t10,c12 isomer. For example, the following U.S. patents all focus primarily on these two isomers: U.S. Pat. Nos. 6,677,470; 6,060,514; 6,015,833; 6,214,372; 6,225,486; 6,410,761; 6,242,621; 6,333,353; 6,465,666; 6,524,527; 6,534,663; 6,534,110; 6,410,078; 6,184,009; 6,160,140; and 6,271,404. However, very few, if any, studies have been conducted using the many other isomers of CLA. Accordingly, what is needed is the discovery of other useful isomers of CLA.

### SUMMARY OF THE INVENTION

The present invention relates to the field of human and animal nutrition, and in particular to certain novel compositions of conjugated linoleic acids (CLA). In particular, the present invention relates to CLA compositions comprising the c10,t12, c10,c12, t9,c11 and/or c9,c11 isomers of conjugated linoleic acid.

Accordingly, in some embodiments, the present invention provides compositions comprising conjugated linoleic acid, the composition comprising at least 1% of at least one of the c10,t12, c10,c12, t9,c11 or c9,c11 isomers of conjugated linoleic acid determined as a percentage of all isomers of conjugated linoleic acid in the composition. In some embodiments, the compositions further comprise the t10,c12 isomer of conjugated linoleic acid. In further embodiments, the compositions further comprise the c9,t11 isomer of conjugated linoleic acid. In some preferred embodiments, the compositions comprise at least 5%, 10%, 20%, 30%, 40% or 50% of at least one of the c10,t12, c10,c12, t9,c11 or c9,c11 isomers of conjugated linoleic acid. In other preferred embodiments, the compositions comprise between about 1% and 90% of the c10,t12, c10,c12, t9,c11 or c9,c11 isomers of conjugated linoleic acid. In still more preferred embodiments, the compositions comprise between about 5% and 60% of the c10,t12, c10,c12, t9,c11 or c9,c11 isomers of conjugated linoleic acid, and even more preferably between about 10% and 35% of the c10,t12, c10,c12, t9,c11 or c9,c11 isomers of conjugated linoleic acid. In some preferred embodiments, the compositions comprise at least 5% of at least one of the c10,t12, c10,c12, t9,c11 or c9,c11 isomers of conjugated linoleic acid. In other preferred embodiments, the compositions comprise between about 1% and 90% of the c10,t12, c10,c12, t9,c11 or c9,c11 isomers of conjugated linoleic acid. In still more preferred embodiments, the compositions comprise between about 10% and 35% of the c10,t12, c10,c12, t9,c11 or c9,c11 isomers of conjugated linoleic acid. In some preferred embodiments, the compositions comprise at least 5% of the c9,t11 isomer of conjugated linoleic acid. In other preferred embodiments, the compositions comprise between about 1% and 90% of the c9,t11 isomer of conjugated linoleic acid. In some embodiments, the compositions further comprise an antioxidant compound. In other embodiments, the compositions comprise less than 100 ppm volatile organic compounds. The present invention is not limited to any particular form of the c10,t12, c10,c12, t9,c11 or c9,c11 isomers. Indeed, the c10,t12, c10,c12, t9,c11 or c9,c11 isomers can be provided in a variety ways, including, but not limited to, a fatty acid, an alkylester, and an acylglyceride.

In some embodiment, the present invention provides food compositions comprising the foregoing c10,t12, c10,c12, t9,c11 or c9,c11 isomer CLA compositions. The present invention is not limited to any particular type of food composition. Indeed, a variety of food compositions are contemplated, including, but not limited to, functional foods, nutritional supplement foods, infant foods, pregnancy foods, or elderly foods.

In some embodiments, the present invention provides pharmaceutical compositions comprising the foregoing c10,t12, c10,c12, t9,c11 or c9,c11 isomer compositions. In further embodiments, the present invention provides nutritional or pharmaceutical compositions comprising the CLA composition of claim 1 and a carrier suitable for oral, intraintestinal, or parenteral administration.

In still further embodiments, the present invention provides acylglycerides having the following structure: 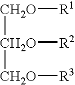
wherein at least two of R1, R2 and R3 are at least one of c10,t12, c10,c12, t9,c11 or c9,c11 conjugated linoleic acyl residues. In some embodiments, the present invention provides a powder comprising at least one type of acylglyceride as previously set forth. In other embodiments, the present invention provides an oil comprising at least one type of acylglyceride as previously set forth. In some embodiments, the oil further comprises an antioxidant. In further embodiments, the present invention provides a food composition comprising at least one type of acylglyceride as previously set forth set forth. The present invention is not limited to any particular type of food composition. Indeed, a variety of food compositions are contemplated, including, but not limited to, functional foods, nutritional supplement foods, infant foods, pregnancy foods, or elderly foods. In still other embodiments, the present invention provides pharmaceutical compositions comprising at least one type of triglyceride as previously set forth. In still further embodiments, the present invention provides nutritional or pharmaceutical compositions comprising at least one type of acylglyceride as previously set forth and a carrier suitable for oral, intraintestinal, or parenteral administration. In some embodiments, the other of R₁, R₂, and R₃ is a t10,c12 conjugated linoleic acyl residue. In some embodiments, the other of R₁, R₂, and R₃ is a c9,t11 conjugated linoleic acyl residue. In some embodiments, the other of R₁, R₂, and R₃ is a medium chain acyl residue. In some embodiments, the other of R₁, R₂, and R₃ is an acyl residue selected from the group consisting of ω3, ω6, and ω9 fatty acyl residues.

In some embodiments, the present invention provides an oil comprising acylglyceride molecules comprising SN1, SN2, and SN3 positions, wherein at least 1% of the SN1, SN2, and SN3 positions are occupied by at least one of c10,t12, c10,c12, t9,c11 or c9,c11 conjugated linoleic acyl residues.

In some embodiments, the present invention provides methods for altering lipid synthesis in a subject comprising: a) providing a subject and a composition comprising at least one of the c10,t12, c10,c12, t9,c11 or c9,c11 isomers of CLA; and b) administering the composition to the subject under conditions such that lipid synthesis is altered. In some embodiments, the composition comprising c10,t12, c10,c12, t9,c11 or c9,c11 CLA comprises at least 5% of the c10,t12, c10,c12, t9,c11 or c9,c11 isomers of conjugated linoleic acid determined as a percentage of all isomers of conjugated linoleic acid in the composition. In some preferred embodiments, the c10,t12, c10,c12, t9,c11 or c9,c11 isomer is provided as a free fatty acid. In other preferred embodiments, the c10,t12, c10,c12, t9,c11 or c9,c11 isomer is provided as an alkylester. In still other embodiments, the c10,t12, c10,c12, t9,c11 or c9,c11 isomer is provided as an acylglyceride. In some embodiments, the subject is a human subject. In some embodiments, the administration is oral. In some embodiments, the c10,t12, c10,c12, t9,c11 or c9,c11 isomer is provided in a food product. In some preferred embodiments, the alteration is partial inhibition of lipid synthesis.

In some embodiments, the present invention provides methods for reducing body fat comprising: a) providing a subject and a composition comprising c10,t12, c10,c12, t9,c11 or c9,c11 CLA; and b) administering the composition comprising c10,t12, c10,c12, t9,c11 or c9,c11 CLA to the subject under conditions such body fat is reduced. In some embodiments, the composition comprising c10,t12, c10,c12, t9,c11 or c9,c11 CLA comprises at least 1% of the c10,t12, c10,c12, t9,c11 or c9,c11 isomers of conjugated linoleic acid determined as a percentage of all isomers of conjugated linoleic acid in the composition. In some preferred embodiments, the composition comprising c10,t12 CLA comprises at least 5%, 10%, 20% or 30% of the c10,t12, c10,c12, t9,c11 or c9,c11 isomer of conjugated linoleic acid determined as a percentage of all isomers of conjugated linoleic acid in the composition. In some embodiments, the composition comprising c10,t12, c10,c12, t9,c11 or c9,c11 CLA comprises between about 5% and 90%, and preferably between about 10% and 35%, of the c10,t12, c10,c12, t9,c11 or c9,c11 isomer of conjugated linoleic acid determined as a percentage of all isomers of conjugated linoleic acid in the composition. In some embodiments, the c10,t12, c10,c12, t9,c11 or c9,c11 isomer is provided as a free fatty acid. In other embodiments, the c10,t12 isomer is provided as an alkylester. In still other embodiments, the c10,t12, c10,c12, t9,c11 or c9,c11 isomer is provided as an acylglyceride. In some embodiments, the subject is a human subject. In some embodiments, the administration is oral. In further embodiments, the c10,t12, c10,c12, t9,c11 or c9,c11 isomer is provided in a food product.

### DESCRIPTION OF THE FIGURES

FIG. 1: Temporal pattern of milk fat content during abomasal infusion of mixtures of conjugated linoleic acid (CLA).

FIG. 2: Temporal pattern of milk fat secretion during abomasal infusion of mixtures of conjugated linoleic acid (CLA).

FIG. 3. Temporal pattern of milk fat yield in cows during abomasal infusions.

FIG. 4. Secretion of milk fatty acids classified by their origin.

### DEFINITIONS

As used herein, “conjugated linoleic acid” or “CLA” refers to any conjugated linoleic acid or octadecadienoic free fatty acid. It is intended that this term encompass and indicate all positional and geometric isomers of linoleic acid with two conjugated carbon-carbon double bonds any place in the molecule. CLA differs from ordinary linoleic acid in that ordinary linoleic acid has double bonds at carbon atoms 9 and 12. Examples of CLA include cis- and trans isomers (“E/Z isomers”) of the following positional isomers: 2,4-octadecadienoic acid, 4,6-octadecadienoic acid, 6,8-octadecadienoic acid, 7,9-octadecadienoic acid, 8,10-octadecadienoic acid, 9,11-octadecadienoic acid and 10,12 octadecadienoic acid, 11, 13 octadecadienoic acid, 12, 14 octadecadienoic acid; 13, 15 octadecadienoic acid; and 15, 17 octadecadienoic acid). As used herein, “CLA” encompasses a single isomer, a selected mixture of two or more isomers, and a non-selected mixture of isomers obtained from natural sources, as well as synthetic and semisynthetic CLA.

As used herein, the term “isomerized conjugated linoleic acid” refers to CLA synthesized by chemical methods (e.g., aqueous alkali isomerization, non-aqueous alkali isomerization, or alkali alcoholate isomerization).

As used herein, the term “conjugated linoleic acid moiety” refers to any compound or plurality of compounds containing conjugated linoleic acids or derivatives. Examples include, but are not limited to fatty acids, alkyl esters, and triglycerides of conjugated linoleic acid.

As used herein, it is intended that “triglycerides” or “acylglycerides” of CLA contain CLA at any or all of three positions (e.g., SN-1, SN-2, or SN-3 positions) on the triglyceride backbone. Accordingly, a triglyceride containing CLA may contain any of the positional and geometric isomers of CLA.

As used herein, it is intended that “esters” of CLA include any and all positional and geometric isomers of CLA bound through an ester linkage to an alcohol or any other chemical group, including, but not limited to physiologically acceptable, naturally occurring alcohols (e.g., methanol, ethanol, propanol). Therefore, an ester of CLA or esterified CLA may contain any of the positional and geometric isomers of CLA.

As used herein, “c” encompasses a chemical bond in the cis orientation, and “t” refers to a chemical bond in the trans orientation. If a positional isomer of CLA is designated without a “c” or a “t”, then that designation includes all four possible isomers. For example, 10,12 octadecadienoic acid encompasses c10,t12; t10,c12; t10,t12; and c10,c12 octadecadienoic acid, while t10,c12 octadecadienoic acid or CLA refers to just the single isomer.

As used herein, the term “oil” refers to a free flowing liquid containing long chain fatty acids (e.g., CLA), triglycerides, or other long chain hydrocarbon groups. The long chain fatty acids, include, but are not limited to the various isomers of CLA.

As used herein, the term “food product” refers to any food or feed suitable for consumption by humans, non-ruminant animals, or ruminant animals. The “food product” may be a prepared and packaged food (e.g., mayonnaise, salad dressing, bread, or cheese food) or an animal feed (e.g., extruded and pelleted animal feed or coarse mixed feed). “Prepared food product” means any pre-packaged food approved for human consumption.

As used herein, the term “foodstuff” refers to any substance fit for human or animal consumption.

As used herein, the term “functional food” refers to a food product to which a biologically active supplement has been added.

As used herein, the term “infant food” refers to a food product formulated for an infant such as formula.

As used herein, the term “elderly food” refers to a food product formulated for persons of advanced age.

As used herein, the term “pregnancy food” refers to a food product formulated for pregnant women.

As used herein, the term “nutritional supplement” refers to a food product formulated as a dietary or nutritional supplement to be used as part of a diet.

As used herein, the term “medium chain fatty acyl residue” refers to fatty acyl residues derived from fatty acids with a carbon chain length of equal to or less than 14 carbons.

As used herein, the term “long chain fatty acyl residue” refers to fatty acyl residues derived from fatty acids with a carbon chain length of greater than 14 carbons.

As used herein, the term “volatile organic compound” refers to any small carbon-containing compound which exists partially or completely in a gaseous state at a given temperature. Volatile organic compounds may be formed from the oxidation of an organic compound (e.g., CLA). Volatile organic compounds include, but are not limited to pentane, hexane, heptane, 2-butenal, ethanol, 3-methyl butanal, 4-methyl pentanone, hexanal, heptanal, 2-pentyl furan, octanal.

As used herein, the term “metal oxidant chelator” refers to any antioxidant that chelates metals. Examples include, but are not limited to lecithin and citric acid esters.

As used herein, the term “alcoholate catalyst” refers to alkali metal compounds of any monohydric alcohol, including, but not limited to, potassium methylate and potassium ethylate.

### DETAILED DESCRIPTION OF THE INVENTION

The present invention relates to the field of human and animal nutrition, and in particular to certain novel compositions of conjugated linoleic acids (CLA). In particular, the present invention relates to CLA compositions comprising the c10,t12, c10,c12, t9,c11 and c9,c11 isomers of conjugated linoleic acid.

The present invention provides compositions comprising at least one of the c10,t12, c10,c12, t9,c11 and c9,c11 isomers of conjugated linoleic acid. The present invention is not limited to compositions comprising any particular amount of these isomers. In some embodiments, the compositions comprise 1%, 5%, 10%, 50%, 90% or more of one of the c10,t12, c10,c12, t9,c11 and c9,c11 isomers of CLA determined as a percentage of total CLA isomers present in the composition. In some embodiments, the isomer is provided as an alkylester, for example, an ethyl, methyl, or propyl ester c10,t12, c10,c12, t9,c11 or c9,c11 CLA. In other embodiments, as described in more detail below, the isomer is provided as part of an acylglyceride molecule. The present invention provides dietary supplements, food supplements, and food products comprising these compositions.

Previously, only the t10,c12 isomer of CLA had been shown to have an inhibitory effect on lipid synthesis in milk fat depression model. Surprisingly, the present inventors found that the c10,t12, c10,c12, t9,c11 and c9,c11 isomers of CLA, isomers that have not been previously shown to have a biological effect, have strong inhibitory effects on lipid synthesis in a milk fat depression model. Thus, the present invention provides methods of inhibiting lipid synthesis in a subject by administering c10,t12, c10,c12, t9,c11 and/or c9,c11 CLA to the subject. In other embodiments, the present invention provides methods of reducing body fat subject by administering c10,t12, c10,c12, t9,c11 and/or c9,c11 CLA to the subject. In still further embodiments the c10,t12, c10,c12, t9,c11 and/or c9,c11 isomer is administered to stimulate the immune system by increasing the number of white blood cells such as natural killer cells.

### I. Synthesis of Compositions Containing the Isomers

The present invention provides compositions comprising c10,t12 conjugated linoleic acids (octadecadienoic acids) and derivatives (e.g., esters, protected acids, acylglycerides, etc.) thereof. In some preferred embodiments, the compositions comprise greater than about 1%, 5%, 10%, 20%, 30%, 50% or 90% c10,t12 CLA or CLA residues determined as a percentage of all isomers of conjugated linoleic acid in the composition.

In some embodiments, the present invention provides methods for producing compositions enriched for the c10,t12 isomer of CLA. In some embodiments, the c10,t12 CLA isomers are prepared from a starting composition of t10,c12 CLA isomers. In preferred embodiments, the starting t10,c12 CLA composition comprises greater than about 50%, 60%, 70%, 80% or 90% t10,c12 CLA. The t10,c12 CLA can be obtained from Natural ASA, Norway, or synthesized according to the methods described in Scholfield and Koritalia, “A Simple Method for Preparation of Methyl trans-10,cis-12 Octadecadienoate,” JOACS 47(8):303 (1970), Berdeau et al., “A Simply Method of Preparation of Methyl trans-10, cis-12- and cis-9, trans-11-Octadecadienoates from Methyl Linoleate,” JAOCS 75:1749-1755 (1998), and U.S. Pat. No. 6,225,486 and related patents, all of which are incorporated herein by reference. In some embodiments, the starting t10,c12 composition is treated by bubbling with nitrogen and then acidified with nitric acid, preferably by addition of about 0.5% to about 50% nitric acid, most preferably about 1% to about 5% nitric acid on a weight/weight basis. In some embodiments, the mixture is incubated at about 60-90° C., most preferably about 80-85° C. for about 3 to 10 hours, preferably about 5-6 hours. In some embodiments, the temperature of the mixture is then increased by about 5° C. to about 30° C., preferably by about 5-10° C., and then mixture incubated for an additional 5-15 hours, preferably about 8-10 hours. In some embodiments, the mixture is then washed a plurality of times with water until the pH reaches approximately 5. In further embodiments, the sample is then dried under a vacuum.

In some embodiments, the dried sample, now comprising a mixture of t10,c12, c10,t12 and t10,t12 CLA isomers, is diluted in a 1 to 10 fold excess, preferably about a four fold excess, of an organic solvent, preferably acetone. In further embodiments, the resulting mixture is incubated at low temperature, about 0oC to about −70° C., preferably about −30° C., for about 10 to 40 hours, preferably about 20 hours. In some embodiments, the resulting phases are separated by filtration. The liquid phase is enriched for c10,t12 CLA. In some embodiments, the solvent is evaporated from the liquid phase and the solution degassed. In further embodiments, the ethyl esters are converted to fatty acids by methods known in the art. In still further embodiments, the fatty acids are distilled under a vacuum.

In some embodiments, the present invention provides methods for producing compositions enriched for the t9,c11 isomer of CLA. In some embodiments, the t9,c11 CLA isomer is prepared from a starting composition of c9,t11 CLA isomer. In preferred embodiments, the starting c9,t11 CLA composition comprises greater than about 50%, 60%, 70%, 80% or 90% c9,t11 CLA. The c9,t11 CLA can be obtained from Natural ASA, Norway, or synthesized according to the methods described in Scholfield and Koritalia, “A Simple Method for Preparation of Methyl trans-10,cis-12 Octadecadienoate,” JOACS 47(8):303 (1970), Berdeau et al., “A Simply Method of Preparation of Methyl trans-10, cis-12- and cis-9, trans-11-Octadecadienoates from Methyl Linoleate,” JAOCS 75:1749-1755 (1998), and U.S. Pat. No. 6,225,486 and related patents, all of which are incorporated herein by reference. In some embodiments, the starting c9,t11 composition is treated by bubbling with nitrogen and then acidified with nitric acid, preferably by addition of about 0.5% to about 50% nitric acid, most preferably about 1% to about 5% nitric acid on a weight/weight basis. In some embodiments, the mixture is incubated at about 60-90° C., most preferably about 80-85° C. for about 3 to 10 hours, preferably about 5-6 hours. In some embodiments, the temperature of the mixture is then increased by about 5° C. to about 30° C., preferably by about 5-10° C., and then mixture incubated for an additional 5-15 hours, preferably about 8-10 hours. In some embodiments, the mixture is then washed a plurality of times with water until the pH reaches approximately 5. In further embodiments, the sample is then dried under a vacuum.

In some embodiments, the dried sample, now comprising a mixture of c9,t11, t9,c11 and t9,t11 CLA isomers, is diluted in a 1 to 10 fold excess, preferably about a four fold excess, of an organic solvent, preferably acetone. In further embodiments, the resulting mixture is incubated at low temperature, about 0° C. to about −70° C., preferably about −30° C., for about 10 to 40 hours, preferably about 20 hours. In some embodiments, the resulting phases are separated by filtration. The liquid phase is enriched for t9,c11 CLA. In some embodiments, the solvent is evaporated from the liquid phase and the solution degassed. In further embodiments, the ethyl esters are converted to fatty acids by methods known in the art. In still further embodiments, the fatty acids are distilled under a vacuum.

### II. General Sources of Conjugated Linoleic Acids

The compositions of the present invention may also preferably contain other isomers of CLA. The conjugated linoleic acid incorporated in these compositions may be made by a variety of methods, for example, those described in U.S. Pat. Nos. 6,015,833 and 6,060,514, each of which is herein incorporated by reference. In some embodiments, sunflower oil, safflower oil, or corn oil are reacted at an ambient pressure under an inert gas atmosphere with an excess of alkali in a high-boiling point solvent, namely propylene glycol at a temperature below the boiling point of the solvent. In some particularly preferred embodiments, sunflower oil, safflower oil, or corn oil are reacted in the presence of an alkali alcoholate catalyst and a small amount of a suitable solvent. As compared to soybean oil, these oils have lower concentrations of undesirable components such as phosphatides and sterols. These undesirable components may contribute to the formation of gums which foul the conjugation equipment and other undesirable polymers.

### A. Isomerization with Propylene Glycol as a Solvent

In some embodiments of the present invention, the conjugated linoleic acid is produced by nonaqueous alkali isomerization. The reaction conditions of the controlled isomerization process allow for precise control of the temperature (and constant ambient pressure) of the conjugation process. Preferably the alkali is an inorganic alkali such as potassium hydroxide, cesium hydroxide, cesium carbonate or an organic alkali such as tetraethyl ammonium hydroxide. The catalyst is preferably provided in a molar excess as compared to the fatty acid content of oil. The solvent is propylene glycol. Preferably, the reaction is conducted within a temperature range 130 to 165° C., most preferably at about 150° C. The time of the reaction may vary, however, there is an increased likelihood of the formation of undesirable isomers when the reaction is conducted for long periods of time. A relatively short reaction time of 2.0 to 6.5 hours has proved satisfactory for excellent yields.

It will be understood to a person skilled in the art that to produce the desired composition, the reaction conditions described above may be varied depending upon the oil to be conjugated, the source of alkali, and equipment. Preanalysis of a particular oil may indicate that the conditions must be varied to obtain the desired composition. Therefore, the temperature range, pressure, and other reaction parameters represent a starting point for design of the individual process and are intended as a guide only. For example, it is not implied that the described temperature range is the only range which may be used. The essential aspect is to provide precise temperature control. However, care must be taken because increasing the pressure may lead to less than complete isomerization and the formation of undesirable isomers. Finally, the length of the conjugation reaction may be varied. Generally, increasing amounts of undesirable isomers are formed with increasing length or reaction time. Therefore, the optimal reaction time allows the reaction to go nearly or essentially to completion but does not result in the formation of undesirable isomers.

Following the conjugation reaction, the resulting CLA containing composition may be further purified. To separate the fatty acids from the conjugation reaction mix, the reaction mix is cooled to approximately 95° C., an excess of water at 50° C. is added, and the mixture slowly stirred while the temperature is reduced to about 50° C. to 60° C. Upon addition of the water, a soap of the fatty acids is formed and glycerol is formed as a by-product. Next, a molar excess of concentrated HCl is added while stirring. The aqueous and nonaqueous layers are then allowed to separate at about 80-90° C. The bottom layer containing water and propylene glycol is then drawn off. The remaining propylene glycol is removed by vacuum dehydration at 60-80° C.

The dried CLA composition may then preferably be degassed in degassing unit with a cold trap to remove any residual propylene glycol. Next, the CLA is distilled at 190° C. in a molecular distillation plant at a vacuum of 10⁻¹ to 10⁻² millibar. The advantage of this purification system is the short time (less than one minute) at which the CLA is held at an elevated temperature. Conventional batch distillation procedures are to be strictly avoided since they involve an elevated temperature of approximately 180-200° C. for up to several hours. At these elevated temperatures the formation of undesirable trans-trans isomers will occur. Approximately 90% of the feed material is recovered as a slightly yellow distillate. The CLA may then be deodorized by heating to about 120°-170° C., preferably at about 150° C. for 2 hours to improve smell and taste. Excessive heat may result in the formation of trans-trans isomers. These procedures produce a CLA composition with a solvent level of less than about 5 ppm, preferably less than about 1 ppm. This process eliminates toxic trace levels of solvent so that the resulting composition is essentially free of toxic solvent residues.

The processes described above are readily adaptable to both pilot and commercial scales. For example, 400 kg of safflower oil may be conjugated at 150° C. for 5 hours in 400 kg of propylene glycol with 200 kg KOH added as a catalyst. The resulting CLA may then be purified as described above. Further, commercial scale batch systems may be easily modified to produce the desired CLA composition. For example, stainless steel reactors should be preferably glass lined to prevent corrosion due to pH levels of below 3.0. However, it should be noted that conjugation processes utilizing nonaqueous solvents are generally less corrosive than those conducted with water.

### B. Isomerization with Alcoholate Catalysts

In other embodiments, the acylglycerides of the present invention incorporate acylglycerides made by the isomerization of linoleic acid in the presence alcoholate catalysts. After fat splitting and dehydration, the free fatty acids are combined with methanol or another monohydric low molecular weight alcohol and heated to the temperature at which the alcohol boils. Esterification proceeds under refluxing conditions with removal of the reaction water through a condenser. After the addition of a further quantity of the same or a different monohydric alcohol an alcoholate catalyst is blended into the ester mix (See, e.g., U.S. Pat. No. 3,162,658, incorporated herein by reference). Typical alcoholate catalysts are sodium or potassium ethoxide, or their methyl, butyl, or propyl counterparts.

In the esterification, methanol or ethanol are preferred, although other branched or straight chain monohydric alcohols may be used. The longer the aliphatic chain of the alkyl group, the more lipid compatible the material becomes. Also the viscosity tends to increase. For different types of feed or food, whose consistency varies, product of varying viscosity can be used to obtain the desired flow or compounding characteristics without affecting the therapeutic or nutritional properties arising from the CLA moieties. The theory and practice of esterification are conventional. A basic explanation of the most common methods is set forth in the McCraw-Hill Encyclopedia of Science & Technology, McGraw-Hill Book Co., New York: 1996 (5th ed.). The animal and human body has a variety of esterases, so that the CLA-ester is cleaved to release the free fatty acids readily. Tissue uptake may have a different kinetics depending on the tissue involved and the benefit sought.

In the isomerization step, it was found that alcoholate catalysis produced a much superior product than aqueous alkali mediated isomerization. The latter process always produced undesirable isomers even under mild reaction conditions. The milder conditions do give lower amounts of unwanted isomers, but at the great expense of yield, as shown in the Examples. In most systems the appearance of the c9,t11 and t10,c12 isomers dominates and they are formed in roughly equimolar amounts. It has not heretofore been possible to control the isomerization of the one isomer to the exclusion of the other. While it is desirable to increase the percentage of one or the other isomer (depending on the physiological effect to be achieved), at present this must largely be carried out by adding an enriched source of the desired isomer.

The preferred starting materials for conjugation with alcoholate catalysts are sunflower oil, safflower oil, and corn oil. Each of these oils contains high levels of linoleic acid and low levels of linolenic acid. Conjugation of linolenic acid results in the formation of several uncharacterized fatty acid moieties, the biological properties of which are unknown. Previous conjugation processes were not concerned with the production of unknown compounds because the products were used in drying oils, paints and varnishes and not in products destined from human or animal consumption.

In some embodiments, it is further contemplated that glycerol and esters of glycerol should be removed before making monoesters of fatty acids. Traces of glycerol present during conjugation contribute to the production of trimethoxypropane and triethoxypropane. Therefore, prior to conjugation, it is preferable to distill monoesters obtained by alcoholysis.

### C. Synthesis of Other CLA Isomers

The present invention also contemplates the synthesis of triglycerides comprising the isomers listed in Table 1 below. In some embodiments of the invention, a partially purified or concentrated isomer of CLA is treated under conditions that cause migration of the double bond system. In preferred embodiments, the conditions comprise heating at least one isomer to about 200-240° C., preferably to about 220° C. In other embodiments, the conditions further comprise reacting the partially purified or concentrated isomer or isomers under nitrogen in a sealed container. Referring to Table 1, the preparations of isomers in column 1 can be used to produce preparations containing a substantial amount of the corresponding isomer in column 2. After the initial conversion reaction, the preparation will contain both the starting isomer and the “sister” isomer. Likewise, the preparations of isomers in column 2 can be used to produce substantial amounts of the corresponding isomer in column 1. The preparations containing both isomers may be further treated to purify the sister isomer (e.g., by gas chromatography). As will be understood by those skilled in the art, it is possible to start with more than one partially purified isomer, thereby producing a preparation containing four, six, eight or more isomers. In further embodiments, a purified preparation of the sister isomer may be prepared by methods known in the art (i.e., gas-liquid chromatography) from the treated preparation containing the initial isomer and its sister isomer.

TABLE 1

| Column 1 | Column 2 |
|---|---|
| c9, t11 | t8, c10 |
| t10, c12 | c11, t13 |
| c7, t9 | t6, c8 |
| t11, c13 | c12, t14 |
| c6, t8 | t5, c6 |
| c5, t7 | t4, c6 |
| c4, t6 | t3, c5 |
| t12, c14 | c13, t15 |
| t13, c15 | c14, t16 |

As demonstrated in the Examples, treatment of purified t10,c12 octadecadienoic acid resulted in the production of c11,t13 octadecadienoic acid. Likewise, concentrated or partially purified c11,t13 octadecadienoic acid can be used to produce t10,c12 octadecadienoic acid.

### D. Other Sources of Conjugated Linoleic Acid Isomers

In other embodiments, the conjugated linoleic acids used to produce the acylglycerides of the present invention are obtained from alternative sources. For example, some isomers (e.g., t10,c12 and c9,t11) are available from commercial sources. In other embodiments, t10,c12 and c9,t11 CLA may be purified by the methods described in Scholfield et al., JAOCS 47(8):303 (1970) and Berdeaux et al., JAOCS 74:1749-55 (1998). This method allows for the crystallization and precipitation of the t10,c12 isomer from a mixture of isomers. If the initial mixture contains predominantly the t10,c12 and c9,t11 isomers (i.e., the isomerization id conducted as described above), then the oil remaining after precipitation will be enriched for c9,t11 CLA. In still further embodiments, the CLA isomers may be prepared by gas chromatography or gas chromatography/mass spectrometry procedures.

### III. Synthesis of Triglycerides

The present invention provides novel acylglycerides containing the c10,t12, c10,c12, t9,c11 and/or c9,c11 isomers of CLA, as well as food compositions, animal feeds, pharmaceutical compositions and nutritional compositions comprising the novel acylglycerides. According to the present invention acylglycerides are provided having the following general structure: 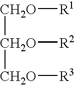
wherein at least 2 of R¹, R² and R³ are c10,t12, c10,c12, t9,c11 and/or c9,c11 acyl residues and the remaining R group is selected from the group consisting of long chain and medium chain fatty acyl residues, ω3, ω6, and ω9 residues, and other conjugated linoleic acyl residues, including, but not limited to the c9,t11 and t10,c12 isomers of conjugated linoleic acid. In some preferred embodiments, the c10,t12 acyl residues occupy the R¹ and R³ positions, while a long chain, medium chain, ω3, ω6, and ω9, c9,t11, t10,c12 or other CLA acyl residue, or combinations thereof, are provided at the R² position. In other preferred embodiments, at least 5%, 10, 20, 305 or 50% of the R¹, R² and R³ (i.e., the SN1, SN2, and SN3) positions are occupied by c10,t12 conjugated linoleic acyl residues. In still other embodiments, a c10,t12 acyl residue is provided at the R¹ position, while a long chain, medium chain, ω3, ω6, and ω9, c9,t11, t10,c12 or other CLA acyl residue, or combinations thereof, are provided at the R¹ and R³ positions.

The present invention is not limited to acylglycerides comprising residues of any particular isomer of conjugated linoleic acid. Indeed, the use of a variety of isomers of conjugated linoleic acid is contemplated, including, but not limited to t10,c12 octadecadienoate; c10,t12 octadecadienoate; c9,t11 octadecadienoate; t9,c11 octadecadienoate; c8,t10 octadecadienoate; t8,c10 octadecadienoate; t11,c13 octadecadienoate; and c11,t13 octadecadienoate, as well as the other isomers listed in Table 1 above.

The present invention is not limited to acylglycerides comprising any particular long chain or medium chain fatty acid residues. Indeed, the incorporation of a variety long chain and medium chain fatty acid residues is contemplated, including, but not limited to decanoic acid (10:0), undecanoic acid (11:0), 10-undecanoic acid (11:1), lauric acid (12:0), cis-5-dodecanoic acid (12:1), tridecanoic acid (13:0), myristic acid (14:0), myristoleic acid (cis-9-tetradecenoic acid, 14:1), pentadecanoic acid (15:0), palmitic acid (16:0), palmitoleic acid (cis-9-hexadecenoic acid, 16:1), heptadecanoic acid (17:1), stearic acid (18:0), elaidic acid (trans-9-octadecenoic acid, 18:1), oleic acid (cis-9-octadecanoic acid, 18:1), nonadecanoic acid (19:0), eicosanoic acid (20:0), cis-11-eicosenoic acid (20:1), 11,14-eicosadienoic acid (20:2), heneicosanoic acid (21:0), docosanoic acid (22:0), erucic acid (cis-13-docosenoic acid, 22:1), tricosanoic acid (23:0), tetracosanoic acid (24:0), nervonic acid (24:1), pentacosanoic acid (25:0), hexacosanoic acid (26:0), heptacosanoic acid (27:0), octacosanoic acid (28:0), nonacosanoic acid (29:0), triacosanoic acid (30:0), vaccenic acid (t-11-octadenecoic acid, 18:1), tariric acid (octadec-6-ynoic acid, 18:1), and ricinoleic acid (12-hydroxyoctadec-cis-9-enoic acid, 18:1).

The present invention is not limited to acylglycerides comprising any particular ω3, ω6, and ω9 fatty acyl residues. Indeed, the present invention encompasses, but is not limited to, acylglycerides including residues of the following ω3, ω6, and ω9 fatty acids:
9,12,15-octadecatrienoic acid (α-linolenic acid) [18:3, ω3];
6,9,12,15-octadecatetraenoic acid (stearidonic acid) [18:4, ω3];
11,14,17-eicosatrienoic acid (dihomo-α-linolenic acid) [20:3, ω3];
8,11,14,17-eicosatetraenoic acid [20:4, ω3],
5,8,11,14,17-eicosapentaenoic acid [20:5, ω3];
7,10,13,16,19-docosapentaenoic acid [22:5, ω3];
4,7,10,13,16,19-docosahexaenoic acid [22:6, ω3];
9,12-octadecadienoic acid (linoleic acid) [18:2, ω6];
6,9,12-octadecatrienoic acid (γ-linolenic acid) [18:3, ω6];
8,11,14-eicosatrienoic acid (dihomo-γ-linolenic acid) [20:3 ω6];
5,8,11,14-eicosatetraenoic acid (arachidonic acid) [20:4, ω6];
7,10,13,16-docosatetraenoic acid [22:4, ω6];
4,7,10,13,16-docosapentaenoic acid [22:5, ω6];
6,9-octadecadienoic acid [18:2, ω9];
8,11-eicosadienoic acid [20:2, ω9]; and
5,8,11-eicosatrienoic acid (Mead acid) [20:3, ω9].

Moreover, acyl residues may be hydroxylated, epoxidated or hydroxyepoxidated acyl residues.

In other embodiments, novel acylglycerides of the present invention are manufactured by using non-specific and position-specific lipases to insert a first fatty acyl residue at position 2 (SN2) of the acylglyceride and a second fatty acyl residue at positions 1 and 3 (SN1 and SN3) of the acylgyceride. Non-specific lipases are lipases that are able to hydrolyse or esterify (i.e., the reverse reaction) fatty acids in all positions on a glycerol. A position-specific or 1,3 specific lipase almost exclusively hydrolyses or esterifies fatty acids in position 1 and 3 on the glycerol backbone. The structured acylglycerides of the present invention are synthesized by first using a non-specifc lipase to attach the desired fatty acid for position 2 to all 3 positions and then hydrolysing the acyl residues in position 1 and 3 using a 1,3 specific lipase. The hydrolysed acids are then removed by distillation before the acids desired to be attached to positions 1 are 3 are added and esterified to position 1 and 3 by the same lipase. The direction of the reaction (hydrolysis or esterification) is easily controlled by water addition or removal respectively. In the following example is a general outline of the method.

In particularly preferred embodiments, a purified aliquot of a first fatty acid (about 3 moles), glycerol (about 1 mole) and up to 10% by weight of acids are mixed with immobilized non-specific lipase (commercially available). The mixture is stirred under vacuum and slightly heated (50-60° C.). The water produced during the esterification is continuously removed by the vacuum suction. After 24-48 hours, the reaction is finished and the enzymes are removed and recovered by filtration. The resulting acylglyceride has the first fatty acid attached at all three positions. The first fatty acid residue at positions 1 and 3 is then removed in by addition of 1,3 specific immobilized lipase (commercially available) and 1% water. The mixture is heated to 50-60° C. and stirred under nitrogen atmosphere for 24-48 hours. The reaction mixture now comprises free fatty acids liberated from position 1 and 3 and monoglycerides (fatty acid B attached to position 2). Next, in preferred embodiments, the fatty acids are distilled off from the mixture by molecular distillation. In further preferred embodiments, about one mole of the monoglyceride is allowed to react for 24-48 hours with 2 moles a second free fatty acid in the presence of 1,3 specific lipase. In some embodiments, this reaction takes place under stirring and vacuum at 50-60° C. to remove water produced in the esterification process. The resulting acylglyceride is a structured triglyceride with the first fatty acid in position 2 and the second fatty acid in positions 1 and 3.

As described above, in some embodiments of the present invention, lipase that specifically acts on the positions 1 and 3 of triglyceride is used as catalyst. The present invention is not limited to the use of any particular 1,3 specific lipase. Examples of 1,3 specific lipases useful in the present invention include lipases produced by a microorganism belonging to the genus Rhizopus, Rhizomucor, Mucor, Penicillium, Aspergillus, Humicola or Fusarium, as well as porcine pancreatic lipase. Examples of commercially available lipases include lipase of Rhizopus delemar (Tanabe Pharmaceutical, Dalipase), lipase of Rhizomucor miehei (Novo Nordisk, Ribozyme IM), lipase of Aspergillus niger (Amano Pharmaceutical, Lipase A), lipase of Humicola lanuginosa (Novo Nordisk, Lipolase), lipase of Mucor javanicus (Amano Pharmaceutical, Lipase M) and lipase of Fusarium heterosporum. These lipases may be used in their native form, or in the form of lipase that has been immobilized on cellite, ion exchange resin or a ceramic carrier.

The amount of water added to the reaction system affects the outcome of the reaction. Transesterification does not proceed in the absolute absence of water, while if the amount of water is too much, hydrolysis occurs, the triglyceride recovery rate decreases, or spontaneous acyl group transfer occurs in a partially acylated glyceride resulting in transfer of the fatty acid at the position 2 to the position 1 or 3. Thus, when using an immobilized enzyme that does not have bonded water, it is effective to first activate the enzyme using a substrate to which water has been added before carrying out the reaction, and then use a substrate to which water is not added during the reaction. In order to activate the enzyme in batch reactions, a substrate containing water at 0 to 1,000% (wt %) of the amount of added enzyme should be used to pretreat the enzyme, and in the case of activating by a column method, a water-saturated substrate should be allowed to continuously flow through the column. The amount of lipase used in a batch reaction may be determined according to the reaction conditions. Although there are no particular limitations on the amount of lipase, 1 to 30% (wt %) of the reaction mixture is suitable when using, for example, lipase of Rhizopus delemar or lipase of Rhizomucor miehei immobilized on cellite or a ceramic carrier.

In some preferred embodiments, the above-mentioned immobilized enzyme can be used repeatedly. Namely, the reaction can be continued by leaving the immobilized enzyme in a reaction vessel after reaction and replacing the reaction mixture with freshly prepared reaction mixture comprising substrate. In addition, for transesterification by a column method, a reaction mixture containing substrate be allowed to flow continuously at the rate of 0.05 to 20 ml/hr per gram of enzyme. In other preferred embodiments, the content of target triglyceride can be increased by performing transesterification repeatedly. Namely, lipase specifically acting on the positions 1 and 3 of the acylglyceride is allowed to act in the presence of the second fatty acid or an ester thereof to obtain a reaction mixture in which fatty acids at positions 1 and 3 are transesterified to the desired fatty acid.

The target acylglycerides of the present invention can easily be isolated by routine methods such as liquid chromatography, molecular distillation, downstream membrane fractionation or vacuum superfractionation or a combination thereof. Purification of the target acylgycerides of the present invention can be performed by alkaline deacidation, steam distillation, molecular distillation, downstream membrane fractionation, vacuum superfractionation, column chromatography, solvent extraction or membrane separation, or a combination thereof so as to remove the above-mentioned fatty acids released by the transesterification and unreacted unsaturated fatty acids.

### IV. Stabilization of CLA Acylglycerides

The present invention also contemplates stabilization of the c10,t12, c10,c12, t9,c11 and/or c9,c11 compositions by preventing oxidation of the compounds. The present invention is not limited to any one mechanism. Indeed, an understanding of the mechanism of the invention is not necessary to produce the composition or perform the methods of the present invention. Nevertheless, unlike non-conjugated fatty acids, CLA does not appear to form stable hydroperoxides as breakdown products as do non-conjugated unsaturated fatty acids. This was demonstrated experimentally by measuring peroxide values (PV) spectrophotometrically by a chlorimetirc ferric thiocyanate method. After storage in open glass, the PV of CLA was 32; in comparison, the value for linoleic acid was 370.

CLA forms volatile organic compounds during breakdown, including hexane. Products stored in a steel drum for several weeks were found to contain up to 25 ppm hexane. Hexane has a characteristic taste and smell that is undesirable in food products. Hexane is a volatile solvent for which an upper limit exists in food laws. Oxidation of CLA appears to be caused by the presence of metal contaminants. Thus, a system for removal of such compounds that promote oxidation during purification is advantageous.

Furthermore, it is also advantageous to add compounds to c10,t12, c10,c12, t9,c11 and/or c9,c11 compositions to decrease oxidation during storage. Compounds that prevent oxidation (antioxidants) have two general mechanisms of action. The first is the prevention of oxidation by lipid peroxide radical scavenging. Examples include but are not limited to tocopherols and ascorbylpalmitate. The second mechanism for preventing oxidation is by the chelation of metal ions. Examples of metal oxidant chelators include, but are not limited to, citric acid esters, EDTA and lecithin. Some commercially available compounds (e.g., Controx, Grumau (Henkel), Illertissen, DE) include both peroxide scavengers and metal chelators (e.g., lecithin, tocopherols, ascorbylpalmitate, and citric acid esters). In some embodiment of the present invention, metal oxidant chelators are added to CLA containing compounds to prevent oxidation. In other embodiments, a combination of metal oxidant chelators and peroxide scavengers is included in the c10,t12, c10,c12, t9,c11 and/or c9,c11 compositions.

In some embodiments, gas chromatography/mass spectroscopy is used in detect the presence of volatile organic breakdown products of CLA. In other embodiments, oil stability index (OSI) measurements are used to detect the presence of volatile organic breakdown products of CLA. In some embodiments of the present invention, pro-oxidants (e.g., iron) are removed from the CLA acylglyceride compositions. Methods for removing pro-oxidants include, but are not limited to, distillation or by adsorption. In some embodiments of the present invention, compounds are added to prevent oxidation of CLA.

In preferred embodiments, precautions are taken during purification to prevent oxidation during storage. These precautions include the removal of compounds that serve as pro-oxidants, including but not limited to iron or other metals. In some embodiments, metals are removed by treating with adsorbing agents, including but not limited to bleaching earth, active charcoal zeolites, and silica. In other embodiments, the pro-oxidants are removed by distillation.

In some embodiments, pro-oxidants are removed in a distillation process. In some preferred embodiments, distillation of c10,t12, c10,c12, t9,c11 and/or c9,c11 compositions of the present invention is performed on a molecular distillation apparatus. Distillation is carried out at 150° C. and a pressure of 10⁻² mbar. The present invention is not intended to be limited to the conditions described for distillation. Other temperatures and pressures are within the scope of the present invention.

In some embodiments, oxidation of the c10,t12, c10,c12, t9,c11 and/or c9,c11 compositions of the present invention is prevented by the addition of metal oxidant chelators or peroxide scavengers to the finished product. In some embodiments, the amount of oxidation is measured by the oil stability index (OSI). The OSI (See e.g., AOCS official method Cd 12b-92) is a measurement of an oil's resistance to oxidation. It is defined mathematically as the time of maximum change of the rate of oxidation. This rate can be determined mathematically. Experimentally, the OSI is calculated by measuring the change in conductivity of deionized water is which volatile organic acids (oxidation products) are dissolved. When performing OSI measurements, it is important to avoid contamination by trace amounts of metals, which can accelerate the oxidation process. This is generally accomplished by careful washing of all glassware used with a cleaning solution lacking chromate or surfactants. Water must be deionized and all solvents must be of a highly purified grade.

### V. Formulation and Administration of c10,t12, c10,c12, t9,c11 and/or c9,c11 Compositions

The c10,t12, c10,c12, t9,c11 and/or c9,c11 compositions of the present invention may be provided in a variety of forms. In some embodiments, administration is oral. The c10,t12, c10,c12, t9,c11 and/or c9,c11 compositions may be formulated with suitable carriers such as starch, sucrose or lactose in tablets, pills, dragees, capsules, solutions, liquids, slurries, suspensions and emulsions. Preferably, the CLA formulations contain antioxidants, including, but not limited to Controx, Covi-OX, lecithin, and oil soluble forms of vitamin C (ascorbyl palmitate). The c10,t12, c10,c12, t9,c11 and/or c9,c11 compositions may be provided in oily solution, or in any of the other forms discussed above. The tablet or capsule of the present invention may be coated with an enteric coating which dissolves at a pH of about 6.0 to 7.0. A suitable enteric coating which dissolves in the small intestine but not in the stomach is cellulose acetate phthalate. In some embodiments, the c10,t12, c10,c12, t9,c11 and/or c9,c11 compositions are provided as soft gelatin capsules containing 10-1500 mg of the desired isomer. The c10,t12, c10,c12, t9,c11 and/or c9,c11 compositions may also be provided by any of a number of other routes, including, but not limited to, intravenous, intramuscular, intra-arterial, intramedullary, intrathecal, intraventricular, transdermal, subcutaneous, intraperitoneal, intranasal, enteral, topical, sublingual or rectal means. Further details on techniques for formulation for and administration and administration may be found in the latest edition of Remington's Pharmaceutical Sciences (Maack Publishing Co., Easton, Pa.).

In particularly preferred embodiments, the c10,t12, c10,c12, t9,c11 and/or c9,c11 compositions of the present invention are combined with an excipient or powdering agent. The mixture is then formed into a powder by methods such as spray drying (See, e.g., U.S. Pat. No. 4,232,052, incorporated herein by reference). In general, spray drying involves liquefying or emulsifying a substance and then atomizing it so that all but a small percentage of water is removed, yielding a free flowing powder. Suitable spray drying units include both high pressure nozzle spray driers and spinning disk or centrifugal spray driers. The present inventors have discovered that powders containing high loads (e.g., 40%-65%) conjugated linoleic acid and/or other oils (e.g., evening primrose oil, borage oil, flax oil, CLA oil) can be formed by the simple spray drying of the emulsion of the oil, excipient and water. It is not necessary to incorporate more complex methods involving spraying into a fluidized bed or spraying in a countercurrent fashion.

The present invention is not limited to any particular excipient. Indeed, a variety of excipients are contemplated, including, but not limited to, HI-CAP 100 (National Starch, Bridgewater, N.J.) and HI-CAP 200 (National Starch, Bridgewater, N.J.). The powder of the present invention contains a high percentage of oil as compared to the excipient. In some embodiments, the oil is 20% of the powder on a weight/weight basis (i.e., the powder contains 20 grams of oil for every 100 grams of powder). In other embodiments, the oil is 35% of the powder on weight/weight basis. In still other embodiments, the oil is at least 50% of the powder on a weight/weight basis. In further embodiments, the oil is at least 60%-65% of the powder on a weight/weight basis. In each case, the oil powder is free flowing and odorless.

An effective amount of a c10,t12, c10,c12, t9,c11 and/or c9,c11 composition may also be provided as a supplement in various food products, including animal feeds, human functional food products, infant food products, nutritional supplements, and drinks. For the purposes of this application, food products containing c10,t12, c10,c12, t9,c11 and/or c9,c11 compositions means any natural, processed, diet or non-diet food product to which exogenous CLA acylglyceride has been added. Therefore, c10,t12, c10,c12, t9,c11 and/or c9,c11 compositions may be directly incorporated into various prepared food products, including, but not limited to diet drinks, diet bars, supplements, prepared frozen meals, candy, snack products (e.g., chips), prepared meat products, milk, cheese, yogurt and any other fat or oil containing foods.

Furthermore, if not properly handled, c10,t12, c10,c12, t9,c11 and/or c9,c11 compositions can contain levels of volatile organic compounds that cause the taste and smell of food products containing the c10,t12, c10,c12, t9,c11 and/or c9,c11 compositions to be adversely effected. It is contemplated that the food products of the present invention that contain c10,t12, c10,c12, t9,c11 and/or c9,c11 compositions having less than 100 ppm volatile organic compounds, and preferably less than 5 ppm volatile organic compounds, are superior in taste and smell to food products containing higher levels of volatile organic compounds and will be preferred in blind taste and smell tests. Accordingly, some embodiments of the present invention provide a food product containing a c10,t12, c10,c12, t9,c11 and/or c9,c11 composition, wherein the conjugated linoleic acid moiety has a sufficiently low volatile organic acid compound concentration so that taste and smell of the food product is not affected.

Use in ruminant feeds requires that c10,t12, c10,c12, t9,c11 and/or c9,c11 is protected against microbial biohydrogenation in the rumen by means of encapsulating the CLA in protective coating or by forming a derivative of the fatty acid. Several methods known in the art.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: M (molar); mM (millimolar); μM (micromolar); kg (kilograms); g (grams); mg (milligrams); μg (micrograms); ng (nanograms); L or l (liters); ml (milliliters); μl (microliters); cm (centimeters); mm (millimeters); nm (nanometers); ° C. (degrees centigrade); KOH (potassium hydroxide); HCL (hydrochloric acid); Hg (mercury).

### EXAMPLE 1

### Synthesis of c10,t12 CLA, t10,c12 and t10,12 Concentrates

This example describes a method for synthesizing compositions enriched for c10,t12 conjugated linoleic acid. Briefly, 1048 g of CLA t10,c12 ethyl esters, 93.8% pure, supplied by Natural ASA, was transferred to a round bottomed flask and bubbled with nitrogen for 15-20 minutes. 21.1 g of nitric acid, 65%, was transferred to same flask under continued nitrogen supply. The mixture was further bubbled with nitrogen for 15 minutes and then heated to 80° C. and kept at 80-85° C. under a nitrogen supply for 5.5 hours and at 85-90° C. for an additional 9 hours. GC-analysis showed an increase in CLA t10,t12 isomer to 64.5%. CLA t10,c12c c10,t12 and c10,c12 were 15.8, 14.1 and 3.5% respectively. The sample was washed several times with water until the pH of the washing water had risen to 5 and then dried under vacuum.

Next, 992 g of the dried sample was diluted in 3970 ml of acetone and placed in a freezer at −30° C. After approximately 20 hours the phases were separated by filtration. GC-analysis of liquid phase showed a reduction of CLA t10,t12 and an increase in the levels of CLA t10,c12 and c10,t12, with the concentration of each of the isomers now being close to 30%. After evaporation of acetone and degassing, free fatty acids were made from the ethyl esters by standard procedures and then distilled under fine vacuum. Fatty acid composition for the final product was 26.5%, 34.9%, 8.3% and 23.0% of the four isomers c10,t12, t10,c12, c10,c12 and t10,t12 respectively. A t10,t12 concentrate was produced as precipitate from crystallization of the nitric acid treated t10,c12 mixture that contained 64.5% of the t,t isomer. The crystals collected at −25 centigrade from an acetone dissolved mixture contained 95.8% t10,t12 CLA. Table 2 presents the isomer content of the composition as determined by gas chromatography.

TABLE 2
Fatty acid composition, c10, t12
Fatty acid composition, % of total fatty acids determined by GC

| Fatty acid concentrate | CLA t10, c12 | CLA c10, t12 | CLA t10, t12 |
|---|---|---|---|
| C16:0 | <0.1 | 0.1 | <0.1 |
| C18:0 | <0.1 | <0.1 | <0.1 |
| C18:1 c9 | 0.9 | 2.6 | <0.1 |
| C18:2 c9, c12 | <0.1 | <0.1 | <0.1 |
| CLA c9, t11 | 4.8 | 2.2 | 0.2 |
| CLA c9, c11 | 0.4 | 0.7 | <0.1 |
| CLA t10, c12 | 91.0 | 34.9 | 1.5 |
| CLA c10, t12 | 0.1 | 26.5 | 1.5 |
| CLA c10, c12 | 0.8 | 8.3 | 0.2 |
| CLA t9, t11 + t10, t12 | 1.3 | 23.0 | 95.8 |
| of which CLA t10, t12 | 0.7 | 22.1 | 91.9 |
| est.* | | | |

*isomers coelutes, estimation based on composition of starting material

### EXAMPLE 2

### Synthesis of t9,c11 Concentrate

1268 grams of a concentrate of c9,t11 CLA (90.1% purity) was heated under stirring and nitrogen atmosphere with 25,52 gram of nitric acid (65%) for four hours at 100 C. After cooling, the mixture was washed several times with water and dried under vacuum at 70 C. 1216 gram of the mixture, now comprising c9,t11, t9,c11 and t9,t11 CLA isomers, was diluted in 8100 ml of acetone and kept at −25 to −28 C for 48 hours before filtration. The crystals were washed two times with cold acetone, and the acetone used for washing was added to the supernatant. The solvent was removed under vacuum and distilled after degassing on a molecular distillation plant at 185 C. The final product contained 32.1% t9,c11 CLA. The product can be purified further by repeated crystallizations. From the precipitate, a t9,t11 was collected and used as a reference in the study of milk fat depression, example 4.

### EXAMPLE 3

### Effect of c10,t12 CLA in a Milk Fat Depression Model

When CLA is prepared with alkaline isomerization of linoleic acid, the isomers t10,c12 and c9,t11 are formed. Further, trans, trans isomers of 9,11 and 10,12 can be formed catalytically at high temperatures, as well as t8,c10 and c11,t13. All c,t forms of these positional isomers have been tested in lipid inhibition models in purified or semipurifed forms, and only t10,c12 has been shown to be an active inhibitor of lipid synthesis. This example describes, for the first time, a biological effect of the c10,t12 isomer of CLA.

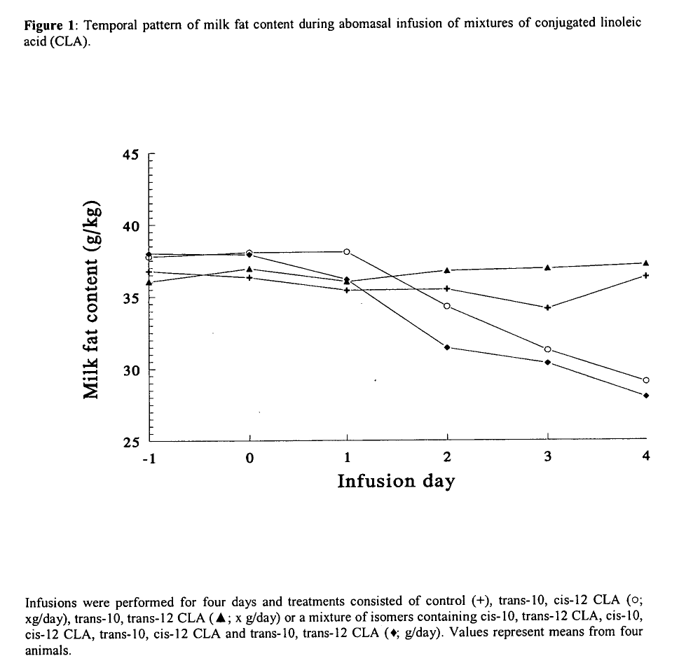

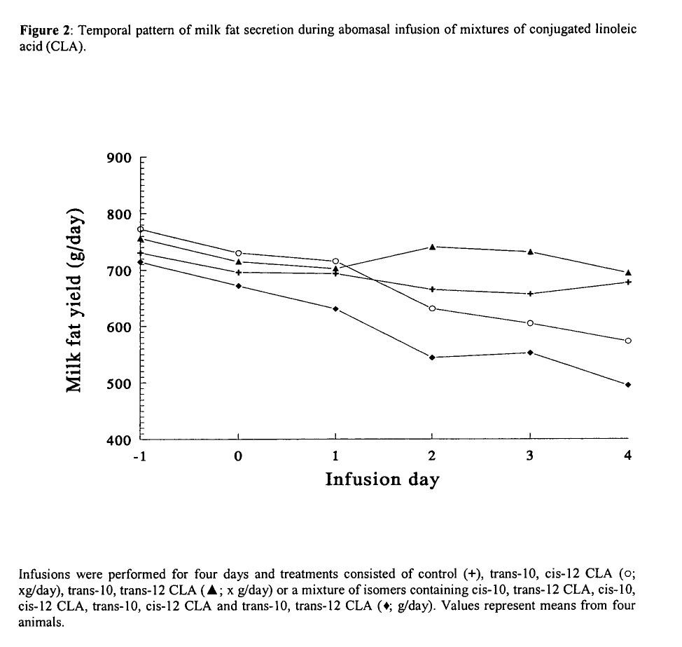

Emulsions of three different isomer preparations (see Table 2; t10,c12; t10,t12; and a mixture of t10, c12; c10,t12 and t10,t12 isomers; prepared as described in Example 1) were prepared essentially the same way as described by Chouinard, et al., J. Nutr. 129:1579 [1999]. The concentration of CLA in these emulsions was 1 g/L and and the emulsions were administered continuously to cows at the rate of 3 1/24 h over four days as described by Chouinard, et al., J. Nutr. 129:1579 [1999]. Briefly, the emulsions were continuously infused into the abomasum via infusion lines that pass through the rumen cannula and omasal canal and peristaltic pumps. Milk was sampled daily and concentration of milk fat was determined by infrared analysis using Milko-Scan 133B analyser (Foss Electric, Hillerød, Denmark). Referring to FIGS. 1 and 2, the isomer mixture was shown to have a similar or slightly stronger effect on milk fat synthesis than did purified t10,c12 (positive control), despite the fact the mixture contained only about 60% of the c10,t12 and t10,c12 isomers. The t10,t12 isomer in purified form did not have any effect on the concentration of milk fat. Therefore, the c10,t12 isomer is a stronger inhibitor of lipid synthesis than t10,c12, which had previously been the only isomer of CLA shown to be active in a milk fat depression model. Table 3 presents the mean treatment effects on intake and milk production.

TABLE 3

| | | | | |
|---|---|---|---|---|
| Control | t10, c12 | t10, t12 | mixture | s.e.m.¹ |

| | | | | | |
|---|---|---|---|---|---|
| Intake (kg DM/day) | | | | | |
| Yield | | | | | |
| Milk (kg/day) | 19.7 | 22.2 | 19.9 | 18.7 | 2.52 |
| Fat (g/day) | 677^{ab} | 573^{ab} | 694^{a} | 495^{b} | 53.8 |
| Protein (g/day) | 734 | 795 | 712 | 689 | 72.4 |
| Lactose (g/day) | 866 | 981 | 895 | 806 | 107.3 |
| Concentration | | | | | |
| Fat (g/kg) | 36.3^{a} | 29.1^{b} | 7.2^{a} | 28.0^{b} | 1.66 |
| Protein (g/kg) | 37.9 | 36.8 | 36.6 | 37.9 | 1.34 |
| Lactose (g/kg) | 43.1^{ab} | 43.6^{ab} | 44.6^{a} | 42.1^{b} | 0.59 |

¹Standard error of the means; error degrees of freedom 6.
^{a,b}Means within row not sharing common superscripts differ significantly (P < 0.05).

### EXAMPLE 4

### Effect of t9,c11 CLA in a Milk Fat Depression Model

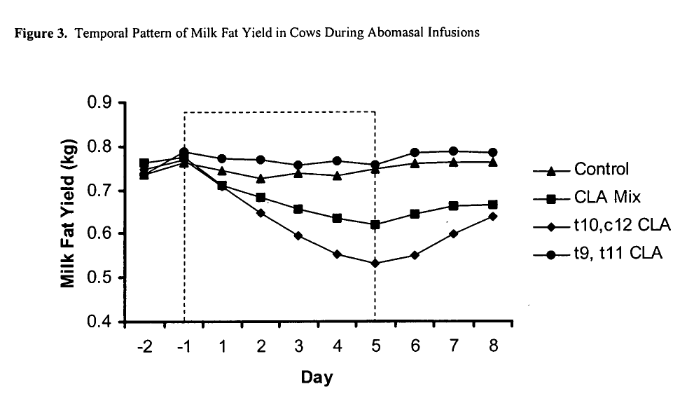

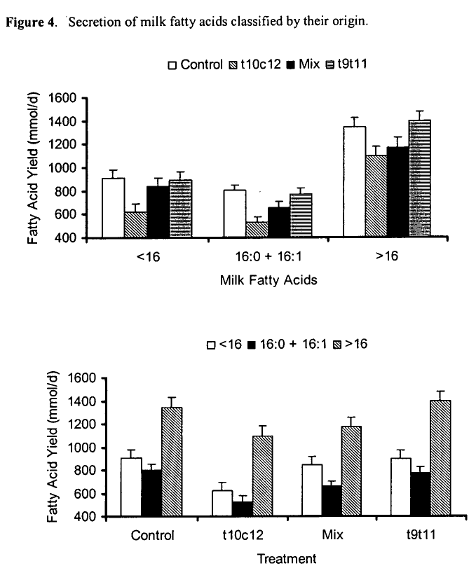

Four rumen-fistulated lactating Holstein cows (149±18 DIM) were randomly assigned in a 4×4 Latin square experiment. Treatments were abomasal infusions of 1) ethanol (control), 2) trans-10, cis-12 CLA supplement (positive control), 3) trans-9, trans-11 CLA supplement, and 4) trans-9, cis-11 CLA supplement. The trans-10, cis-12 and trans-9, trans-11 CLA supplements were of high purity (>90%), whereas the trans-9, cis-11 CLA supplement consisted mainly of 3 CLA isomers: trans-9, cis-11 (32%), cis-9, trans-11 (29%) and trans-9, trans-11 (17%). CLA supplements supplied 5 g/d of the CLA isomer of interest and the daily dose was provided by infusion at 6 h intervals. Treatment periods were 5 d in length with a 7 d washout interval. Milk yield and DMI were unaffected by treatment (P>0.05). Milk fat yield was reduced 27% by the trans-10, cis-12 CLA treatment and 15% by the trans-9, cis-11 CLA treatment, while the trans-9, trans-11 CLA treatment had no effect (P<0.001). Milk protein content and yield were reduced by the trans-9, trans-11 CLA treatment only (P<0.01). The transfer efficiency of specific CLA isomers within respective treatment groups was 22% for trans-10, cis-12 CLA, 21% for trans-9, trans-11 CLA and 46% for trans-9, cis-11 CLA (P<0.001). Overall, abomasal infusion of trans-9, cis-11 CLA reduced milk fat synthesis, but to a lesser extent than trans-10, cis-12 CLA. This indicates that trans-9, cis-11 CLA may be responsible for a portion of the decreased milk fat production in some situations of diet-induced-MFD. The data are summarized in Tables 4 and 5 and FIGS. 3 and 4.

TABLE 4
Fatty acid profiles of the conjugated linoleic acid (CLA)
supplements and amounts of fatty acids infused.

| | Treatment | | |
|---|---|---|---|
| Fatty acid | t10, c12 CLA | t9, c11 CLA | t9, t11 CLA |
| Composition, % | | | |
| 16:0 | <0.1 | <0.1 | <0.1 |
| 18:0 | <0.1 | <0.1 | <0.1 |
| 18:1 cis-9 | 0.6 | 7.0 | <0.1 |
| 18:2 cis-9, cis-12 | <0.1 | 0.1 | <0.1 |
| Conjugated linoleic acid | | | |
| C18:2 cis-9, trans-11 | 3.8 | 28.8 | 0.3 |
| C18:2 trans-9, cis-11 | — | 32.1 | 0.6 |
| C18:2 trans-10, cis-12 | 93.3 | 4.9 | <0.1 |
| C18:2 cis-9, cis-11 | — | 8.2 | 0.4 |
| C18:2 trans-9, trans-11* | — | 16.6 | 98.0 |
| Other | 2.3 | 0.9 | <0.3 |
| Abomasal infusion, g/d | | | |
| 16:0 | <0.1 | <0.1 | <0.1 |
| 18:0 | <0.1 | <0.1 | <0.1 |
| cis-9 18:1 | <0.1 | 1.1 | <0.1 |
| cis-9, cis-12 18:2 | <0.1 | <0.1 | <0.1 |
| Conjugated linoleic acid | | | |
| C18:2 cis-9, trans-11 | 0.2 | 4.5 | <0.1 |
| C18:2 trans-9, cis-11 | — | 5.0 | <0.1 |
| C18:2 trans-10, cis-12 | 5.0 | 0.8 | <0.1 |
| C18:2 cis-9, cis-11 | — | 1.3 | <0.1 |
| C18:2 trans-9, trans-11 | — | 2.6 | 5.0 |
| Other | 0.1 | 0.1 | <0.1 |

TABLE 5
Performance of Lactating Dairy Cows during Abomasal Infusions.¹

| | Treatment | | | | | |
|---|---|---|---|---|---|---|
| | | t9, c11 | t10, c12 | t9, t11 | | |
| Variable | Control | CLA | CLA | CLA | SEM | P² |
| DM intake, | 19.1 | 19.6 | 18.3 | 18.2 | 0.6 | NS |
| kg/d | | | | | | |
| Milk, kg/d | 20.6 | 20.4 | 21.4 | 19.1 | 0.8 | NS |
| Milk fat | | | | | | |
| % | 3.60^{b} | 3.07^{c} | 2.54^{d} | 4.03^{a} | 0.15 | <0.001 |
| kg/d | 0.740^{a} | 0.628^{b} | 0.541^{c} | 0.770^{a} | 0.028 | <0.001 |
| Milk protein | | | | | | |
| % | 3.01^{a} | 3.01^{a} | 3.02^{a} | 2.87^{b} | 0.13 | <0.001 |
| kg/d | 0.617^{a} | 0.615^{a} | 0.648^{a} | 0.547^{b} | 0.043 | <0.01 |
| Milk lactose | | | | | | |
| % | 4.76^{a} | 4.64^{b} | 4.68^{b} | 4.78^{a} | 0.06 | <0.01 |
| kg/d | 0.97 | 0.94 | 1.00 | 0.92 | 0.04 | NS |

¹Values represent an average of day 4 and 5 of supplementation.
²Statistical probability of treatment effects. Means within a row with different superscripts differ (P < 0.05).

What should be clear from above is that the present invention provides novel compositions comprising the c10,t12 and/or t9,c11 isomers of CLA which can be used in pharmaceutical compositions, animal feeds and in products suitable for human consumption. All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in medicine, biochemistry, or related fields are intended to be within the scope of the following claims.

1. A composition comprising conjugated linoleic acid, said composition comprising at least 1% of an isomer of conjugated linoleic acid selected from the group consisting of c10,t12 conjugated linoleic acid, c10,c12 conjugated linoleic acid, t9,c11 conjugated linoleic acid, and c9,c11 conjugated linoleic acid, said percentage determined as a percentage of all isomers of conjugated linoleic acid in the composition.

2. The composition of claim 1, further comprising the t10,c12 isomer of conjugated linoleic acid.

3. The composition of claim 1, further comprising the c9,t11 isomer of conjugated linoleic acid.

4. The composition of claim 1, wherein said composition comprises at least 5% of an isomer of conjugated linoleic acid selected from the group consisting of c10,t12 conjugated linoleic acid, c10,c12 conjugated linoleic acid, t9,c11 conjugated linoleic acid, and c9,c11 conjugated linoleic acid.

5. The composition of claim 1, wherein said composition comprises between about 1% and 99% of an isomer of conjugated linoleic acid selected from the group consisting of c10,t12 conjugated linoleic acid, c10,c12 conjugated linoleic acid, t9,c11 conjugated linoleic acid, and c9,c11 conjugated linoleic acid.

6. The composition of claim 1, further comprising an antioxidant compound.

7. The composition of claim 1, wherein said composition comprises less than 100 ppm volatile organic compounds.

8. The composition of claim 1, wherein said isomer of conjugated linoleic acid is a free fatty acid.

9. The composition of claim 1, wherein said isomer of conjugated linoleic acid is an alkylester.

10. The composition of claim 1, wherein said isomer of conjugated linoleic acid is present in an acylglyceride molecule.

11. A food composition comprising the CLA composition of claim 1.

12. The food composition of claim 11, wherein said food composition is a functional food, nutritional supplement food, infant food, pregnancy food, or elderly food.

13. A pharmaceutical composition comprising the CLA composition of claim 1.

14. A nutritional or pharmaceutical composition comprising the CLA composition of claim 1 and a carrier suitable for oral, intraintestinal, or parenteral administration.

15. An acylglyceride having the following structure: 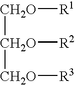
wherein at least two of R1, R2 and R3 are selected from the group consisting of an isomer of conjugated linoleic acid selected from the group consisting of c10,t12 conjugated linoleic acid, c10,c12 conjugated linoleic acid, t9,c11 conjugated linoleic acid, and c9,c11 conjugated linoleic acid acyl residues.

16. A powder comprising at least one type of acylglyceride as set forth in claim 15.

17. An oil comprising at least one type of acylglyceride as set forth in claim 15.

18. The oil of claim 17, wherein said oil further comprises an antioxidant.

19. A food composition comprising at least one type of acylglyceride as set forth in claim 15.

20. The food composition of claim 19, wherein said food composition is a functional food, nutritional supplement food, infant food, pregnancy food, or elderly food.

21. A pharmaceutical composition comprising at least one type of triglyceride as set forth in claim 15.

22. A nutritional or pharmaceutical composition comprising at least one type of acylglyceride as set forth in claim 15 and a carrier suitable for oral, intraintestinal, or parenteral administration.

23. The acylglyceride of claim 15, wherein the other of R₁, R₂, and R₃ is a t10,c12 conjugated linoleic acyl residue.

24. The acylglyceride of claim 15, wherein the other of R₁, R₂, and R₃ is a c9,t11 conjugated linoleic acyl residue.

25. The acylglyceride of claim 15, wherein the other of R₁, R₂, and R₃ is a medium chain acyl residue.

26. The acylglyceride of claim 15, wherein the other of R₁, R₂, and R₃ is an acyl residue selected from the group consisting of ω3, ω6, and ω9 fatty acyl residues

27. An oil comprising acylglyceride molecules comprising SN1, SN2, and SN3 positions, wherein at least 1% of said SN1, SN2, and SN3 positions are occupied by an acyl residue of an isomer of conjugated linoleic acid selected from the group consisting of c10,t12 conjugated linoleic acid, t9,c11 conjugated linoleic acid, and c9,c11 conjugated linoleic acid.

28. A method of altering lipid synthesis in a subject comprising:
a) providing a subject and a composition comprising of an isomer of conjugated linoleic acid selected from the group consisting of c10,t12 conjugated linoleic acid, c10,c12 conjugated linoleic acid, t9,c11 conjugated linoleic acid, and c9,c11 conjugated linoleic acid; and
b) administering said composition to said subject under conditions such that lipid synthesis is altered.

29. The method of claim 28, wherein said composition comprises at least 5% of an isomer of conjugated linoleic acid selected from the group consisting of c10,t12 conjugated linoleic acid, t9,c11 conjugated linoleic acid, and c9,c11 conjugated linoleic acid determined as a percentage of all isomers of conjugated linoleic acid in the composition.

30. The method of claim 29, wherein said isomer is provided as a free fatty acid.

31. The method of claim 29, wherein said isomer is provided as an alkylester.

32. The method of claim 29, wherein said isomer is provided as an acylglyceride.

33. The method of claim 28, wherein said subject is a human subject.

34. The method of claim 28, wherein said administration is oral.

35. The method of claim 29, wherein said isomer is provided in a food product.

36. The method of claim 28, wherein said alteration is partial inhibition of lipid synthesis.

37. A method of reducing body fat comprising:
a) providing a subject and a composition comprising of an isomer of conjugated linoleic acid selected from the group consisting of c10,t12 conjugated linoleic acid, c10,c12 conjugated linoleic acid, t9,c11 conjugated linoleic acid, and c9,c11 conjugated linoleic acid; and
b) administering said composition comprising c10,t12 CLA to said subject under conditions such body fat is reduced.

38. The method of claim 37, wherein said composition comprising c10,t12 CLA comprises at least 1% of an isomer of conjugated linoleic acid selected from the group consisting of c10,t12 conjugated linoleic acid, c10,c12 conjugated linoleic acid, t9,c11 conjugated linoleic acid, and c9,c11 conjugated linoleic acid determined as a percentage of all isomers of conjugated linoleic acid in the composition.

39. The method of claim 38, wherein said isomer is provided as a free fatty acid.

40. The method of claim 37, wherein said isomer is provided as an alkylester.

41. The method of claim 37, wherein said isomer is provided as an acylglyceride.

42. The method of claim 37, wherein said subject is a human subject.

43. The method of claim 37, wherein said administration is oral.

44. The method of claim 37, wherein said isomer is provided in a food product.
